(19) United States
(12) Patent Application Publication
(10) Pub. No.: US 2025/0336522 A1
(43) Pub. Date: Oct. 30, 2025

(54) SYSTEMS AND METHODS FOR CONDITION IDENTIFICATION USING ATTENTION-BASED MULTI-MODAL GRAPH

(71) Applicant: Optum, Inc., Minnetonka, MN (US)

(72) Inventors: Arun Kumar TIWARI, New Delhi (IN); Amardeep SHARMA, Delhi (IN); Manish Chandra GUPTA, Greater Noida West (IN); KM Garima MISRA, Mohammadi (IN); Akhil SRIVASTAVA, Greater Noida West (IN); Ashutosh SINGH, Greater Noida (IN)

(21) Appl. No.: 18/646,993

(22) Filed: Apr. 26, 2024

(51) Int. Cl. G16H 50/20; G16H 10/60

(52) U.S. Cl. CPC G16H 50/20; G16H 10/60

(57) ABSTRACT

Systems and methods are disclosed for condition identification. One or more processors may receive a member data object with indicators and dimensions, access a member-specific graph network with nodes representing attributes and weighted edges indicating associations, modify the nodes and edges based on the member data object, generate a multi-modal graph database by combining the modified member-specific graph network and a disease graph network, apply the multi-modal graph database to an attention-based graph neural network (GNN) that identifies associations between nodes by dynamically allocating attention weights to edges, generate an embedding data object with node identifiers and vectors representing features and relationships, select a target node associated with condition data, apply the embedding data object to a classification layer that outputs predicted conditions for the target node, and generate the probability of predicted conditions appearing in the target node.

### TECHNICAL FIELD

The present disclosure relates generally to the technical field of data analytics, predictive analytics, and machine learning. More particularly, the present disclosure relates to adaptation of machine learning techniques for predicting rare conditions associated with an entity.

### BACKGROUND

In the context of data-driven condition identification, the identification of a condition based on generated or received data may prove elusive when endeavoring to identify a rare condition. Conventional techniques in condition identification often misidentify the condition, leading to a delay in identifying the condition, which may involve repeated collection of data, inappropriate actions based on misidentified conditions, and delay in taking appropriate actions for the actual condition. These prolonged timelines can lead to resource inefficiencies and harmful actions being taken due to condition misidentification, impacting the treatment of the condition.

Existing methodologies for rare condition identification face challenges in arriving at accurate condition identification based on encounter-isolated assessments of data. These methods predominantly rely on a singular entity, such as a user, assessing the member based data collected during one or more encounters. Due the condition being a rare condition, the user may initially identify more common conditions, and in some cases may incorrectly act upon the misidentified condition. While the user may be a specialist in specific conditions, a rare condition may elude a specialist that is not trained for that specific rare condition. As a result, a member with a rare condition may not be accurately identified for months or years, as various entities collect data about the member and attempt to correctly identify the condition mainly through trial and error, leading to inefficiencies in resource management for condition identification and treatment.

This disclosure is directed to addressing the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure addresses the technical problem(s) described above or elsewhere in the present disclosure and improves the state of data incident response techniques.

In some aspects, the techniques described herein relate to a computer-implemented method including; receiving, by one or more processors, a member data object associated with a member, the member data object including one or more indicators, each indicator having an associated dimension; accessing, by the one or more processors, a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modifying, by the one or more processors, one or more of the plurality of nodes and one or more edges therebetween of the member-specific graph network based on the one or more indicators and the one or more respective dimensions of the member data object; generating, by the one or more processors, a multi-modal graph database by combining the modified member-specific graph network and a disease graph network; applying, by the one or more processors, the multi-modal graph database to an attention-based graph neural network (GNN) trained to identify associations between nodes by learning to allocate attention weights to edges of the multi-modal graph database; generating, by the one or more processors and based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database; selecting, by the one or more processors, a target node from the plurality of nodes, the target node associated with condition data; applying, by the one or more processors, the embedding data object to a classification layer, the classification layer trained to output one or more predicted conditions for the target node based on one or more input embeddings; and generating, by the one or more processors and based on the applying of the embedding object to the classification layer, a probability of one or more predicted conditions appearing in the target node.

In some aspects, the techniques described herein relate to a system including memory and one or more processors communicatively coupled to the memory, the one or more processors configured to: receive a member data object associated with a member, the member data object including one or more indicators, each indicator having an associated dimension; access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modify one or more of the plurality of nodes and one or more edges therebetween of the member-specific graph network based on the one or more indicators and the one or more respective dimensions of the member data object; generate a multi-modal graph database by combining the modified member-specific graph network and a disease graph network; apply the multi-modal graph database to an attention-based graph neural network (GNN) trained to identify associations between nodes by learning to allocate attention weights to edges of the multi-modal graph database; generate, based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database; select a target node from the plurality of nodes, the target node associated with condition data; apply the embedding data object to a classification layer, the classification layer trained to output one or more predicted conditions for the target node based on one or more input embeddings; and generate, based on the applying of the embedding object to the classification layer, a probability of one or more predicted conditions appearing in the target node.

In some aspects, the techniques described herein relate to one or more non-transitory computer-readable storage media including instructions that, when executed by one or more processors, cause the one or more processors to: receive a member data object associated with a member, the member data object including one or more indicators, each indicator having an associated dimension; access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modify one or more of the plurality of nodes and one or more edges therebetween of the member-specific graph network based on the one or more indicators and the one or more respective dimensions of the member data object; generate a multi-modal graph database by combining the modified member-specific graph network and a disease graph network; apply the multi-modal graph database to an attention-based graph neural network (GNN) trained to identify associations between nodes by learning to allocate attention weights to edges of the multi-modal graph database; generate, based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database; select a target node from the plurality of nodes, the target node associated with condition data; apply the embedding data object to a classification layer, the classification layer trained to output one or more predicted conditions for the target node based on one or more input embeddings; and generate, based on the applying of the embedding object to the classification layer, a probability of one or more predicted conditions appearing in the target node.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1A is a diagram showing an example of a system environment, according to some embodiments of the disclosure.

FIG. 1B is a diagram of example components of a condition identification platform, according to some embodiments of the disclosure.

FIG. 1C is a diagram of example components of a condition identification module, according to some embodiments of the disclosure.

FIG. 2 is a flowchart showing a method for condition identification, according to some embodiments of the disclosure.

FIG. 3 is a diagram of example components of a member graph network, according to some embodiments of the disclosure.

FIG. 4 is a diagram of example components of a disease graph network, according to some embodiments of the disclosure.

FIG. 5 is a diagram of example components of an attention framework, according to some embodiments of the disclosure.

FIG. 6 shows an example machine-learning training flow chart, according to some embodiments of the disclosure.

FIG. 7 illustrates an implementation of a computer system that executes techniques presented herein, according to some embodiments of the disclosure.

### DETAILED DESCRIPTION

The present disclosure relates generally to the technical field of data analytics, predictive analytics, and machine learning. This disclosure encompasses techniques for enhancing identification of rare conditions in entities (e.g., patients). Specifically, it introduces systems and methods leveraging machine learning and rules-based approaches to analyze patient-specific and condition-specific data collected over time, in order to make accurate and timely predictions of rare conditions.

Traditional approaches in identifying rare conditions often struggle with a phenomenon known as diagnostic odyssey. A diagnostic odyssey refers to the prolonged and often complex process of seeking a definitive diagnosis for a patient's symptoms, typically involving multiple medical tests, consultations with various specialists, and sometimes years of uncertainty. This journey can be particularly challenging for patients with rare or undiagnosed conditions, as they navigate through the healthcare system in search of answers and effective treatments.

Conventional methods typically rely on doctor diagnosis, which oftentimes seek to rule out more common conditions before progressing to referrals to specialists. Further, conventional methodologies fall short of leveraging broad diagnostic data of patients with similar conditions to identify potential rare conditions of the patient. Typically, rare conditions are not even considered until well after the patient is far along in a diagnostic odyssey. Such limitations can lead to inefficiencies, unnecessary resource consumptions, and reduced effectiveness in identifying rare conditions in patients.

To address these concerns, the present disclosure provides systems and methods to refine and enhance the datasets as well as data analytical and computational techniques used to identify rare conditions. The techniques provided in the present disclosure leverage machine-learning (e.g., neural-networks), specifically attention-based graph neural networks (GNNs), to identify rare conditions in patients and suggest clinical tests to confirm these rare conditions. By employing attention-based GNNs, the systems and methods identify rare conditions by analyzing patient-specific characteristics collected through the patient's experience and exposures over multiple encounters, which may span over multiple lab tests, imaging, treatments, vitals, providers, and the like.

The disclosed technique results in a number of technical advantages in at least several technical fields, including but not limited to data analytics, predictive analytics, artificial intelligence, business intelligence, and data visualization. The disclosed technique implements attention-based GNNs, where both the patient data and drug-disease-symptom data are represented in graph networks of nodes and edges that represent discrete characteristics or attributes and relationships therebetween. The patient data graph network and drug-disease-symptom graph network are combined and applied to a trained attention-based GNN, which modifies the importance of specific attributes of the various nodes and the weights of the edges between nodes. The resultant output is a data object which predicts a classification of an unknown node within the graph network, such as a disease node. The prediction can be designed to be specific to rare diseases and conditions. The technique is implemented at every new encounter of the patient and, as more data is collected during the patient's journey, the system begins identifying potential rare conditions and suggesting clinical tests to confirm the rare conditions, such as genetic testing. Through the use of graph networks representing relationships between various patient attributes or characteristics and the application of such networks to an attention-based GNN configured to adaptively learn and update graph structures, the disclosed technique enables more precise targeting and selection of proper tests to conduct on the patient to diagnose rare conditions considerably earlier in the diagnostic process, reducing or even eliminating diagnostic odyssey. By detecting potential rare conditions early in the diagnostic process, the disclosed technique reduces overall resource utilization to arrive at a proper diagnosis and allows earlier treatment for patients with a rare condition.

The technical improvements and advantages discussed above are not the sole improvements and advantages, and additional technical improvements and advantages will be discussed in the following sections. Further, based on the present disclosure, other technical improvements and advantages will be apparent to one of ordinary skill in the art.

By way of example, in a practical application of the system, consider a scenario where a patient has an undiagnosed rare condition, such as Meniere's disease. Meniere's disease is a rare, progressive, chronic condition of the inner ear which worsens over time without proper treatment. Oftentimes, Meniere's disease is a diagnosis of last resort-all other potential diagnoses have been ruled out. The patient with the undiagnosed condition goes to their primary care doctor and a standard battery of tests are run for vertigo and hearing loss. The resulting data, along with patient-specific data and subjective data such as their indicated level of hearing loss or vertigo, are provided to the system in the form of a data object. A patient graph network is updated by applying this data to update relevant nodes within the patient graph network that are associated with the relevant data types. This graph network, along with a drug-disease-symptom-protein network based on broadly available drug interaction and symptom data, are applied to an attention-based GNN trained to modify weights within the combined network to find associations between patient conditions, attributes, and the like, and one or more rare disease. At this first encounter, the system does not output the likelihood of a rare condition. The patient then has a second encounter, this time with a thyroid specialist, where additional tests are performed due to identification of elevated thyroid levels from test in the initial encounter. These additional results are provided to the system, which re-runs the entire process and this time provides an output that indicates the patient has a high likelihood of having Meniere's disease. Based on this, the patient is referred to hearing specialist, where specific tests for Meniere's are performed and a diagnosis is made. Without the system, it is likely the patient would have had dozens of additional encounters and tests, over the course of years, before a similar diagnosis occurred. This example demonstrates how the system's graph neural network strategies effectively reduce diagnostic odyssey.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

Various non-limiting embodiments of the present disclosure will now be described to provide an overall understanding of the principles of the structure, function, and use of systems and methods disclosed herein for data environment management.

Reference to any particular activity is provided in this disclosure only for convenience and not intended to limit the disclosure. A person of ordinary skill in the art would recognize that the concepts underlying the disclosed devices and methods may be utilized in any suitable activity. For example, while the present disclosure is in the context of condition identification, one of ordinary skill would understand the applicability of the described systems and methods to similar tasks in a variety of contexts or environments. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Training the machine-learning model may include one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-Prototypes or K-Means may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. After training the machine-learning mode, the machine-learning model may be deployed in a computer application for use on new input data that it has not been trained on previously.

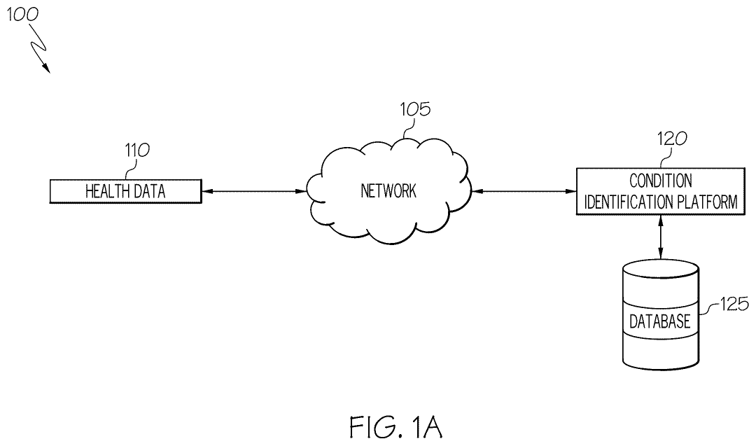

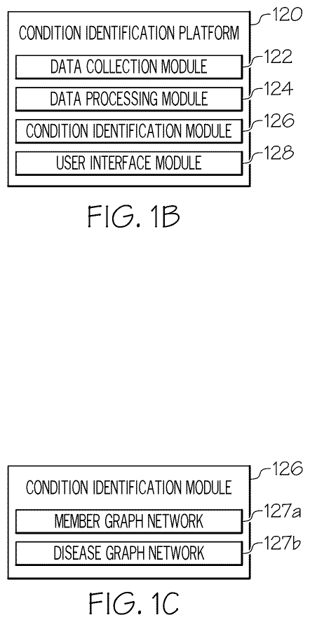

FIG. 1A illustrates a diagram of a system designed for the integration and analysis of health data networks, in accordance with certain embodiments of the present disclosure. The depicted environment, labeled as environment 100, is consistent with a specific embodiment of this disclosure. Environment 100 encompasses a communication infrastructure termed as network 105, which facilitates connectivity to various health data 110 sources, and further integrates with a condition identification platform 120 that incorporates a comprehensive database 125. This database 125 is structured to store and manage a Weighted Network of Drug-Disease-Symptom data alongside Patient's Weighted Electronic Health Records (EHR) network, embodying a rich dataset where weights represent various metrics such as intensity and likelihood of symptoms, or dosage efficacy of drugs for specific disease stages.

In some embodiments, various components within environment 100 interact via network 105. Network 105 facilitates communication between the condition identification platform 120 and other systems, including one or more systems such as health data 110. Health data 110 may contain data, data entries, and/or data objects relevant to health-related and operational activities within the health data integration and analysis environment. Network 105 may encompass various types of networks, including, but not limited to, data networks, wireless networks, telephony networks, or any combination thereof, to support robust and secure data exchange across environment 100. Within environment 100, any of these components, including health data 110 sources, condition identification platform 120, and one or more additional systems, may communicate with one another based on established access permissions.

Any of the health data 110 sources, the database 125, and/or one or more other systems associated with the condition identification platform 120 may contain a diverse collection of structured and/or unstructured data pertinent to health records, treatment outcomes, patient interactions, medication efficacy, and operational processes within the healthcare environment. In some embodiments, this data, organized into one or more data objects, spans a variety of dimensions including patient health records, treatment records, medication administration logs, API request and response data related to health data exchanges, security and compliance documentation, along with insights from health data analytics. This extensive repository, which includes health records, patient treatment activities, medication effectiveness data, and compliance statuses, may be stored in storage solutions that range from local to cloud-based data storage systems, ensuring secure storage and accessibility for ongoing processing and health data analytical evaluation.

The database 125 may support the storage and retrieval of data related to one or more datasets and/or data objects, such as patient health records, medication administration logs, and API request and response data related to health data exchanges, storing metadata and operational data about one or more entities represented in these datasets, as well as any information received from the condition identification platform 120. Database 125 may comprise one or more systems, including but not limited to a relational database management system (RDBMS), a NoSQL database, or a graph database, tailored to meet the specific needs and use cases within environment 100, particularly for managing the complex and interconnected data of the healthcare domain.

In some embodiments, database 125 may embody any type of database system, including relational, hierarchical, object-oriented, among others, where data is systematically arranged in structures such as tables, graphs, or other suitable formats. Database 125 is configured to store and facilitate retrieval of data utilized by the condition identification platform 120, encompassing information such as patient health records, Drug-Disease-Symptom relationships, and outcomes generated by the platform. Furthermore, database 125 is adapted to maintain a vast array of information, for instance, to aid in the analysis, prediction, and management of patient health outcomes within environment 100.

In some embodiments, database 125 comprises a machine learning-based analytics database that outlines relationships, associations, and connections between input parameters derived from health data and output parameters representing various health-related metrics for condition identification and prediction. This includes leveraging machine learning algorithms aimed at learning mappings between data inputs (e.g., symptom intensity, medication dosage, patient attributes) and outputs such as disease prediction accuracy, treatment effectiveness, and symptom-disease mappings. This analytics database is designed to be periodically updated to incorporate additional insights obtained through continuous machine learning processes.

Condition identification platform 120 interacts with other components within network 105 utilizing established or evolving communication protocols. These protocols ensure efficient interactions between various nodes within the network and dictate the conventions for creating, sending, and interpreting data exchanges across communication links. They are operational across different layers, from generating physical signals to facilitating specific software applications engaged in data transmission or reception, thereby enabling robust and secure data flow within environment 100.

Communications between the various components of the networks are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers.

In operation, environment 100 serves as a platform for processing and analyzing transaction data within health data networks, utilizing techniques such as data analytics, artificial intelligence, and database management. For instance, in an embodiment, environment 100 facilitates the generation of insights, metrics, and data objects from various datasets, including patient and drug data, according to predefined criteria or multiple parameters.

To fulfill these functions, the condition identification platform 120 may utilize one or more methodologies, such as the deployment of a machine-learning model within the condition identification module 126, specifically designed to analyze health data to uncover patterns, trends, and/or anomalies across environment 100. Moreover, the condition identification platform 120 leverages the data collection module 122 and the data processing module 124 to aggregate and refine health-related data, including symptoms, drug efficacies, and disease correlations for advanced analysis.

For optimized data storage and retrieval, the database 125 is capable of archiving metadata associated with health data, encompassing information on data sources, types, and structures. This database 125 further maintains records on the insights generated by the condition identification platform 120, such as disease-symptom relationships, treatment outcomes, and statistical health data.

Beyond the analysis of health data, environment 100 facilitates a variety of applications, from data visualization and search functionalities to predictive modeling. For instance, environment 100 enables practitioners or researchers to query health data for specific indicators that match given criteria, or to visualize health statistics through dynamic graphs and charts.

FIG. 1B is a diagram illustrating example components of the condition identification platform 120, in accordance with some embodiments. In some embodiments, condition identification platform 120, as part of environment 100, is configured to analyze diverse datasets, such as health data, and generating data objects, including insights and metrics pertinent to patient health and treatment outcomes. The terms “component” or “module” within this depiction are inclusive of both hardware and software elements implemented via a processor or comparable technology. Notably, the condition identification platform 120 comprises modules dedicated to the collection, processing, analysis of health data, and the generation of informative data objects. These encompass the data collection module 122, the data processing module 124, the condition identification module 126, and the user interface module 128. The architecture provides versatility in the configuration of these modules, allowing for the integration of their functions into a consolidated framework or the distribution across various modules with akin functionalities.

In some embodiments, the data collection module 122 of the condition identification platform 120 is tasked with the acquisition of data from one or more sources and in one or more formats during the functioning of one or more systems of environment 100. This module is configured to manage one or more data types, including, but not limited to, electronic health records, patient-reported symptoms, medication and treatment data, diagnostic data, analytics data, drug data, and the like. It is also configured to handle proprietary or generated data such as health analytics, risk assessments, and outcomes from predictive modeling.

The data is ingested into the system via multiple pathways, thus providing flexibility in the collection mechanism for the condition identification platform 120. One such pathway involves an Application Programming Interface (API) that establishes a secure communication channel for automated data transfer between the data collection module 122 and external health data 110 sources, enabling real-time or batch-based data acquisition. An alternative pathway permits manual input by authorized personnel through a dedicated user interface module 128, where input methods include file uploads or direct data entry into predefined fields. Furthermore, data intake can be facilitated through third-party integrations, middleware, or direct database queries aimed at populating database 125. The data collection module 122 also implements data validation and integrity checks to ensure the accuracy and reliability of the ingested data.

In some embodiments, the data processing module 124 of the condition identification platform 120 is configured to process and prepare the collected data for further analysis by the condition identification module 126. The data processing module 124 is configured to augment and/or cleanse the data, removing irrelevant or redundant information, and/or converting the data into a format that is amenable for analysis by the condition identification module 126. This module is configured to refine the initial data collection, transforming raw, heterogeneous data into a standardized, uniform format for downstream analysis. The data processing module 124 utilizes a variety of algorithms for data standardization, thereby addressing discrepancies in data types, units, or terminologies emanating from diverse sources.

Additionally, the data processing module 124 incorporates error-handling mechanisms configured to identify and amend potential inaccuracies or anomalies within the data. These mechanisms may include rule-based checks, probabilistic data matching, or data imputation techniques, which are all targeted at preserving the quality and integrity of the data. The data processing module 124 also supports parallel processing capabilities, allowing for the concurrent handling of multiple data streams. This feature is particularly advantageous for processing large volumes of data or enabling real-time analytics.

Upon receiving the processed data from the data processing module 124, the condition identification module 126 is configured to apply this data within a structured framework designed to facilitate advanced health condition identification and analysis. This module leverages the capabilities of graph neural networks (GNNs) to interpret and analyze the complex relationships inherent in health data, including but not limited to patient symptoms, medication efficacy, disease correlations, and treatment outcomes. The condition identification module 126 systematically organizes the data into one or more of a member graph network 127a and/or a disease graph network 127b (FIG. 1C) and/or a multi-modal graph database, enabling the dynamic representation of various health-related entities and their interconnections.

In some embodiments, the condition identification module 126 utilizes an attention-based GNN architecture to assign variable importance to the edges connecting nodes within the graph, allowing for a nuanced understanding of the relationships between different health data points. Furthermore, the condition identification module 126 is equipped with machine learning algorithms capable of inferring missing or incomplete data within the graph, employing techniques such as weight prediction on edges where information is absent or sparse. The condition identification module 126 is configured to integrate and analyze heterogeneous data sources, from electronic health records (EHRs) to biomedical literature, by creating a comprehensive, interconnected graph network. This integrative approach enables the exploration of potential new correlations and insights into disease mechanisms, drug interactions, and symptom presentations that may not be evident from isolated data points, and enables predictions of conditions based on one or more member data objects.

In some embodiments, the condition identification module 126 is configured to execute one or more queries and/or one or more modifications against one or more graph network, such as one or more of member graph network 127a, disease graph network 127b, or one or more generated multi-modal graph database, facilitating the identification of potential health conditions, prediction of disease progression, and suggestion of personalized treatment plans (such as genetic testing) based on the analyzed data. By harnessing the computational power of GNNs and the rich dataset within the multi-modal graph database, the module provides a powerful tool for enhancing patient care and advancing medical research.

In some embodiments, FIG. 1C illustrates a schematic representation of the condition identification module 126, according to some embodiments of the disclosure. In some embodiments, the condition identification module 126 includes a member graph network 127a, a disease graph network 127b, and may include one or more additional components to support one or more operational objectives of the condition identification module, such as a generated multi-model graph network and/or database.

In some embodiments, the member graph network 127a is configured to construct a detailed representation of a patient's health profile by organizing data into a structured graph network. This network integrates nodes representing various health-related attributes such as symptoms reported during medical encounters, medications prescribed, and diagnosed conditions. Each node within this network is connected to one or more other and/or adjacent nodes through edges that signify the relationship between these attributes. For instance, an edge might connect a symptom node to a condition node to indicate that the symptom is indicative of the condition. These connections are not merely binary but are enriched with weights, vectors in nature, that quantify aspects such as the intensity of a symptom or the frequency of medication usage, thereby providing a nuanced view of the patient's health status. The member graph network 127a is configured to be updated contemporaneously with information from temporally relevant member data, such as data collected from a recent visit to a healthcare service provider. Thus, in some embodiments, the member graph network 127a is personalized to an individual member when updated with relevant data for that member.

In some embodiments, the disease graph network 127b is configured to map the interrelations between diseases, drugs, and symptoms on a global scale. Similar to the member graph network 127a, the disease graph network 127b employs nodes and weighted edges to represent and quantify relationships. The scope of the disease graph network 127b encompasses an array of medical knowledge including, but not limited to, the efficacy of drugs in treating specific diseases at various stages, symptom prevalence within diseases, the likelihood of disease co-occurrence, and the like. Weights within this graph may take the form of tensors, encapsulating multidimensional data such as drug dosage requirements for different stages of a disease, thereby offering a comprehensive global view of health dynamics. The disease graph network 127b is updated periodically, and includes data which represents current medical research and knowledge as it relates to one or more conditions. In some embodiments, the data within the disease graph network 127b is updated less frequently than the data within the member graph network 127a, and the data within member graph network 127a is updated more frequently than the data within the disease graph network 127b.

The integration of the member graph network 127a and the disease graph network 127b within the condition identification module 126 facilitates a holistic approach to health condition identification and analysis. By merging patient-specific data with global health information, the module is configured to leverage deep insights into disease mechanisms, symptomatology, and treatment efficacy. This integrated approach supports a wide range of applications, from personalized medicine to epidemiological research, by enabling predictions of unknown, and in some cases rare, conditions, prediction of disease progression, and optimization of treatment strategies based on individual patient profiles and broader health data analytics.

Additionally, the condition identification module 126 is configured to utilize advanced graph neural network (GNN) techniques for further analysis and insight generation. These techniques include, but are not limited to, entity resolution to address node ambiguity by merging duplicate nodes across the member graph network 127a and the disease graph network 127b, and weight prediction algorithms to estimate missing edge weights, thereby enriching the graph with previously unavailable data.

In some embodiments, one or both of the member graph network 127a and the disease graph network 127b may be configured as multi-modal networks. In some embodiments, the multi-modality of both the member graph network 127a and the disease graph network 127b is exemplified by their configuration to intake and process not just textual data but also image data, comprehensively capturing and representing health-related information. This capability facilitates the incorporation of diverse data types—from textual descriptions of symptoms, medication names, and diagnostic codes to imaging studies such as X-rays, MRIs, and photographs of dermatological conditions. By doing so, these networks are configured to harness the depth and breadth of available health data, enriching the graph with layers of information that capture the complexity of medical diagnostics and treatments. The integration of both text and image data enables a more holistic view of patient health and disease mechanisms, leveraging the strengths of each data type to improve the accuracy of disease prediction, diagnosis, and treatment planning.

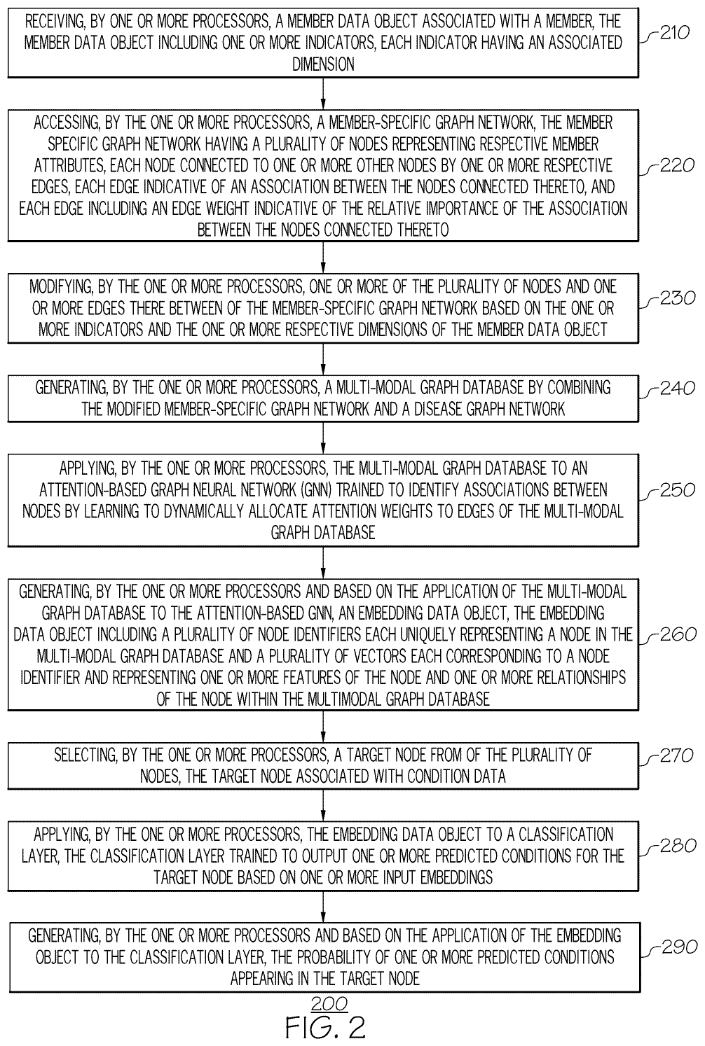

FIG. 2 is a flowchart showing a computer-implemented method for condition identification. In one embodiment, method 200 is performed by the condition identification platform 120, or the components/modules implemented therein. At step 210, the method includes receiving, by one or more processors, a member data object associated with a member, the member data object including one or more indicators, each indicator having an associated dimension. In some embodiments, the indicators within the member data object are health-related, having been collected during a member data collection event, such as a visit to a healthcare provider. These health-related indicators can take various forms, including text data and/or image data. The text data may encompass elements such as doctor's notes, patient-reported symptoms, medical history details, and more. The image data, on the other hand, could include items like X-rays, MRI scans, CT scans, ultrasounds, or other visual medical data collected from the member.

In some embodiments, the dimensions associated with each indicator provide additional context about the nature and specifics of the data point. For a text-based symptom indicator, the associated dimension might include details like the severity of the symptom, the duration for which it has been experienced, or the frequency of occurrence. For an image-based indicator, the dimension could include information about the body part imaged, the imaging technique used, the date of imaging, or any abnormalities noted by medical professionals.

In some embodiments, at step 220, the method 200 includes accessing, by the one or more processors, a member-specific graph network 127a. The member-specific graph network 127a includes a plurality of nodes representing respective member attributes. Each node is connected to one or more other nodes by one or more respective edges, with each edge indicative of an association between the nodes connected thereto. Additionally, each edge includes an edge weight indicative of the relative importance of the association between the nodes connected thereto. The member-specific graph network 127a is configured to capture and represent various aspects of a member's health data, such as encounters, symptoms, providers, medications, conditions, and attributes, in a structured and interconnected manner.

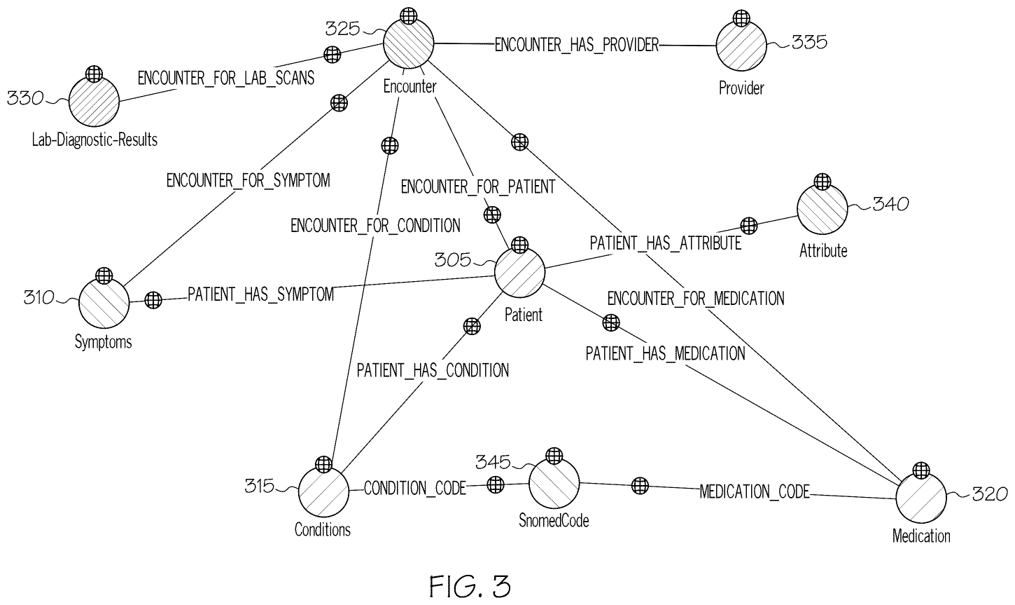

In some embodiments, the member-specific graph network 127a is constructed based on data obtained from various sources, such as electronic health records (EHRs), claims data, patient-reported outcomes, and other administrative data sources. The nodes in the member-specific graph network 127a represent different entities or attributes associated with the member, such as specific encounters, symptoms reported by the member during those encounters, healthcare providers, medications prescribed to the member, diagnosed conditions, and various member attributes like demographics, lifestyle factors, social determinants of health, and the like. The edges connecting these nodes represent the relationships or associations between them, such as the occurrence of a symptom during a particular encounter, the prescription of a medication by a specific provider, or the diagnosis of a condition based on certain symptoms. The member graph network will be discussed later in more detail in relation to FIG. 3.

In some embodiments, the edge weights in the member-specific graph network 127a are assigned based on the strength or significance of the associations between the connected nodes. These weights can be determined using various techniques, such as statistical analysis, domain knowledge, or machine learning algorithms. For example, the weight of an edge connecting a symptom node to an encounter node may represent the intensity or frequency of the symptom reported by the member during that encounter. Similarly, the weight of an edge connecting a medication node to a condition node may indicate the effectiveness or prevalence of using that medication to treat the specific condition. The edge weights are configured based on the relative importance of the associations within the member-specific graph network 127a.

In some embodiments, at step 230, the method 200 includes modifying, by the one or more processors, one or more of the plurality of nodes and one or more edges therebetween of the member-specific graph network 127a based on the one or more indicators and the one or more respective dimensions of the member data object. This modification process includes updating and/or augmenting the member-specific graph network 127a to incorporate new information from the member data object, which includes health-related indicators collected during a member data collection event. The indicators can comprise various types of data, such as text data (e.g., clinical notes, patient-reported outcomes) and image data (e.g., medical imaging scans, photographs).

In some embodiments, the modification of the member-specific graph network 127a involves adding new nodes and edges, updating existing node attributes, and/or adjusting edge weights based on the indicators and their associated dimensions. For example, if the member data object contains information about a new symptom reported by the member during an encounter, a new symptom node may be added to the graph network and connected to the relevant encounter node with an appropriate edge weight. Alternatively, an existing node representative of symptoms may be updated to include the new symptom reported by the member during the encounter. Similarly, if the member data object provides updated information about the severity or frequency of an existing symptom, the corresponding symptom node's attributes and edge weights may be modified accordingly. The dimensional information associated with each indicator, such as the time of occurrence, intensity, or context, is used to guide the modification process and ensure that the member-specific graph network 127a accurately reflects the member's current health status.

In some embodiments, the method 200 includes accessing a disease graph network 127b. The disease graph network 127b is a comprehensive knowledge base that represents the complex relationships between diseases, symptoms, and treatments. It is constructed using information from various sources, such as medical literature, clinical guidelines, expert knowledge, and observational data. The disease graph network 127b consists of nodes representing diseases, symptoms, and treatments, with edges connecting these nodes to indicate their associations. For example, an edge between a disease node and a symptom node may signify that the symptom is commonly observed in patients with that disease, while an edge between a disease node and a treatment node may indicate that the treatment is recommended or frequently prescribed for that disease.

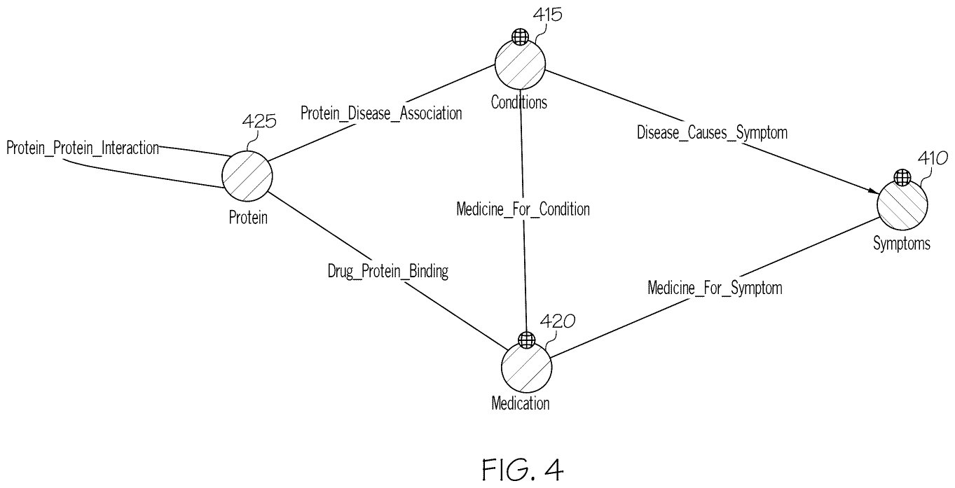

In some embodiments, the edges in the disease graph network 127b are weighted to reflect the strength or importance of the associations between the connected nodes. These edge weights can be derived from various sources, such as the prevalence of a symptom in a specific disease, the effectiveness of a treatment for a particular condition, or the level of evidence supporting the association. The edge weights in the disease graph network 127b are represented as tensors, which are multidimensional arrays that can capture complex relationships and dependencies between the connected nodes. For example, a tensor-based edge weight between a disease node and a symptom node may represent the likelihood and intensity of the symptom occurring at different stages of the disease progression. Similarly, a tensor-based edge weight between a disease node and a treatment node may capture the recommended dosage or duration of the treatment based on the severity or stage of the disease. By incorporating tensor-based edge weights, the disease graph network 127b can provide a more detailed and nuanced representation of the relationships between diseases, symptoms, and treatments, enabling more accurate and personalized insights for condition identification and management. The disease graph network will be discussed later in more detail in relation to FIG. 4.

In some embodiments, prior to modifying the member-specific graph network 127a, the method 200 includes preprocessing, by the one or more processors, the member data object and the disease graph network data. Preprocessing can involve various techniques, such as data normalization, entity resolution, and missing data imputation, to ensure data quality and consistency. Data normalization techniques, such as scaling or standardization, may be applied to ensure that the indicators and their dimensions are represented in a uniform format across different data sources. Entity resolution techniques, such as deduplication or record linkage, may be employed to identify and merge duplicate or related nodes across the member-specific graph network 127a and the disease graph network. Missing data imputation techniques, such as interpolation or machine learning-based approaches, may be used to estimate missing values for indicators or edge weights, enabling a more complete representation of the member's health data in the modified member-specific graph network 127a.

In some embodiments, at step 240, the method 200 includes generating, by the one or more processors, a multi-modal graph database by combining the modified member-specific graph network 127a and a disease graph network 127b. The multi-modal graph database integrates the personalized health information from the member-specific graph network with the general medical knowledge from the disease graph network, creating a comprehensive and heterogeneous representation of the member's health status in the context of known disease associations.

In some embodiments, the combining process involves merging the modified member-specific graph network 127a and the disease graph network 127b. The merging process includes identifying common nodes that are present in both graph networks, such as diseases, symptoms, and treatments. For each common node, the edge information from the modified member-specific graph network 127a and the disease graph network 127b is combined, preserving the unique associations and edge weights from each source. This combination process allows for the integration of personalized health data with general medical knowledge, providing a more comprehensive view of the member's health status.

In some embodiments, the merging process also involves aggregating the node features of each common node from the modified member-specific graph network 127a and the disease graph network 127b using an aggregation function. The aggregation function may vary depending on the type of node and the nature of the features being combined. For example, for a common symptom node, the aggregation function may involve taking the maximum intensity value from the member-specific graph network 127a and combining it with the general likelihood or prevalence information from the disease graph network 127b. For a common treatment node, the aggregation function may involve combining the prescribed dosage or duration information from the member-specific graph network 127a with the recommended dosage range or treatment guidelines from the disease graph network 127b.

In some embodiments, the merging process preserves non-common nodes and their associated edge information from either the modified member-specific graph network 127a or the disease graph network 127b in the merged graph database. Non-common nodes are nodes that are present in only one of the graph networks and do not have a corresponding node in the other graph network. Preserving these non-common nodes and their associated edges maintains the unique information and associations from each source graph network. For example, a specific symptom node that is present in the member-specific graph network 127a but not in the disease graph network 127b may represent a rare or atypical symptom experienced by the member, and preserving this node and its edges in the merged graph database ensures that this valuable information is not lost during the merging process.

In some embodiments, the method 200 utilizes a graph neural network (GNN)-based entity resolution technique to resolve node ambiguity during the merging process of the modified member-specific graph network 127a and the disease graph network 127b. Entity resolution is the process of identifying and merging duplicate or semantically equivalent nodes across different graph networks. In the context of combining the member-specific graph network 127a and the disease graph network 127b, entity resolution is configured such that nodes representing the same entity, such as a specific disease or symptom, are properly merged and not treated as separate entities in the resulting multi-modal graph database.

In some embodiments, the GNN-based entity resolution technique employs similarity metrics to identify and merge duplicate nodes across the modified member-specific graph network 127a and the disease graph network 127b. The similarity metrics may consider various factors, such as the node attributes, the neighborhood structure, and the edge weights, to determine the likelihood of two nodes representing the same entity. For example, two disease nodes with similar names, associated symptoms, and connected treatments are likely to be duplicate representations of the same disease and should be merged in the multi-modal graph database.

In some embodiments, the GNN-based entity resolution technique learns a node embedding representation that captures the semantic similarity between nodes across the graph networks. The node embeddings are generated by training a GNN model on the graph networks, which learns to encode the node features and neighborhood information into a low-dimensional vector space. The similarity between two nodes can then be computed by measuring the distance or similarity between their respective node embeddings. Nodes with high embedding similarity are considered potential duplicates and are merged in the multi-modal graph database.

In some embodiments, the GNN-based entity resolution technique also considers the edge information and topology of the graph networks during the merging process. The technique may analyze the neighborhood structure and the types of edges connecting the nodes to determine the semantic equivalence of nodes across the graph networks. For example, if two symptom nodes have similar neighboring disease nodes and edge types, they are more likely to represent the same symptom and should be merged in the multi-modal graph database.

In some embodiments, at step 250, the method 200 includes applying, by the one or more processors, the multi-modal graph database to an attention-based graph neural network (GNN) trained to identify associations between nodes by learning to dynamically allocate attention weights to edges of the multi-modal graph database. The attention-based GNN is, in some embodiments, a deep learning model that can effectively process and analyze the complex relationships and dependencies within the multi-modal graph database.

In some embodiments, the attention-based GNN employs a self-attention mechanism to learn the importance of different edges in the multi-modal graph database. The self-attention mechanism allows the GNN to dynamically assign attention weights to the edges based on their relevance and significance in capturing the associations between nodes. By learning to allocate higher attention weights to more informative and discriminative edges, the attention-based GNN can focus on more influential relationships and dependencies within the multi-modal graph database, enabling more accurate and efficient node classification and condition identification.

In some embodiments, the attention-based GNN is configured to perform weight prediction on missing edge weights within the multi-modal graph database. During the merging process of the modified member-specific graph network 127a and the disease graph network 127b, there may be instances where edge weights are missing or unavailable due to incomplete or inconsistent data. The attention-based GNN can leverage the available edge weights and the graph structure to estimate or predict the missing edge weights, improving the overall completeness and reliability of the multi-modal graph database.

In some embodiments, the weight prediction process in the attention-based GNN includes employing a loss function based at least in part on one or more default and absent edge weights. The loss function is designed to guide the learning process of the GNN by quantifying the discrepancy between the predicted edge weights and the ground truth or expected edge weights. The loss function may incorporate default edge weights, which are predefined or heuristically assigned weights for missing edges based on domain knowledge or statistical assumptions. Additionally, the loss function may consider absent edge weights, which correspond to edges that are explicitly known to be missing or irrelevant in the multi-modal graph database.

In some embodiments, the loss function in the attention-based GNN is formulated as a weighted combination of different loss terms, such as the reconstruction loss, the regularization loss, the sparsity loss, or the like. The reconstruction loss measures the difference between the predicted edge weights and the available ground truth edge weights, encouraging the GNN to accurately estimate the missing edge weights. The regularization loss imposes constraints on the learned attention weights to prevent overfitting and ensure the generalizability of the GNN. The sparsity loss promotes the learning of sparse attention weights, encouraging the GNN to focus on the most informative and discriminative edges while suppressing less relevant ones.

In some embodiments, the attention-based GNN iteratively updates the edge weights in the multi-modal graph database based on the learned attention weights and the weight prediction process. The GNN propagates the node features and edge information through the graph structure, aggregating the relevant information from neighboring nodes and edges to refine the node representations and edge weights. The updated edge weights reflect the learned importance and relevance of the associations between nodes, providing a more accurate and informative representation of the multi-modal graph database.

By applying the multi-modal graph database to an attention-based GNN with weight prediction capabilities, the method 200 can effectively capture and analyze the complex relationships and dependencies within the integrated health data. The attention-based GNN learns to allocate attention weights to the most informative and discriminative edges, enabling the identification of influential associations between diseases, symptoms, treatments, and other relevant entities. The weight prediction process ensures the completeness and reliability of the multi-modal graph database, even in the presence of missing or incomplete edge information.

In some embodiments, at step 260, the method 200 includes generating, by the one or more processors and based on the application of the multi-modal graph database to the attention-based GNN, an embedding data object. The embedding data object includes a plurality of node identifiers, each uniquely representing a node in the multi-modal graph database, and a plurality of vectors, each corresponding to a node identifier and representing one or more features of the node and one or more relationships of the node within the multi-modal graph database.

In some embodiments, the embedding data object is generated by the attention-based GNN through a process called node embedding. Node embedding is a technique that learns a low-dimensional vector representation for each node in the multi-modal graph database, capturing the node's features and its relationships with other nodes. The attention-based GNN uses the learned attention weights and the aggregated node information to generate these embeddings, which encapsulate the relevant information about each node in a compact and informative manner.

In some embodiments, the node identifiers in the embedding data object serve as unique references to the corresponding nodes in the multi-modal graph database. Each node identifier is associated with a specific node and allows for easy retrieval and mapping of the node's information. The node identifiers can be generated using various techniques, such as hashing the node's attributes or assigning sequential numeric identifiers based on the order of the nodes in the multi-modal graph database.

In some embodiments, the vectors in the embedding data object correspond to the learned node embeddings generated by the attention-based GNN. Each vector is associated with a specific node identifier and represents the node's features and relationships. The dimensions of the vector capture different aspects of the node's characteristics and its interactions with other nodes in the multi-modal graph database. For example, some dimensions of the vector may represent the node's intrinsic features, such as the type of entity (e.g., disease, symptom, treatment), while other dimensions may capture the node's relationships with its neighbors, such as the strength or type of the edges connecting them.

In some embodiments, the node embeddings in the embedding data object are learned by the attention-based GNN through an iterative process of message passing and aggregation. The GNN propagates information across the multi-modal graph database, allowing nodes to update their representations based on the information received from their neighbors. The attention mechanism in the GNN helps to prioritize the most relevant and informative neighbors during this process, ensuring that the node embeddings capture the most significant relationships and dependencies within the graph.

In some embodiments, the node embeddings in the embedding data object are further refined and optimized using various techniques, such as regularization and normalization. Regularization techniques, such as L1 or L2 regularization, can be applied to the embedding vectors to prevent overfitting and ensure the generalizability of the learned representations. Normalization techniques, such as batch normalization or layer normalization, can be used to stabilize the training process and improve the convergence of the attention-based GNN.

In some embodiments, the embedding data object serves as a compact and informative representation of the multi-modal graph database, capturing one or more features and relationships of the nodes. The embedding vectors can be used for various downstream tasks, such as node classification, link prediction, or clustering, enabling efficient and accurate analysis of the health data.

In some embodiments, at step 270, the method 200 includes selecting, by the one or more processors, a target node from the plurality of nodes in the multi-modal graph database. The target node is associated with condition data and represents one or more conditions or diseases for which the likelihood is to be predicted. In the context of condition identification, the target node is typically a condition node in the multi-modal graph database. By selecting the condition node as the target node, the method 200 aims to infer the likelihood of one or more particular condition based on the overall structure and weights of the combined graph. The selection of the condition node as the target node focuses the subsequent prediction and classification tasks on determining the probability of one or more condition given the available evidence and relationships in the multi-modal graph database. It will be appreciated that similar techniques may be employed to predict characteristics of one or more additional nodes within the multi-modal graph neural network, such as predicting which symptoms may be present.

In some embodiments, at step 280, the method 200 includes applying, by the one or more processors, the embedding data object to a classification layer. The classification layer is trained to output one or more predicted conditions for the target node based on the input embeddings. The classification layer takes the embedding vector corresponding to the target node as input and processes it through a series of transformations and activations to generate the predicted conditions.

In some embodiments, the classification layer is trained using labeled data, where each training sample consists of an embedding vector and its corresponding ground truth condition. The training process involves optimizing the weights of the classification layer to minimize a loss function, such as cross-entropy loss or focal loss, which measures the discrepancy between the predicted conditions and the true conditions. In some embodiments, the classification layer is designed to handle multi-class classification, where the target node can be associated with multiple conditions simultaneously. In this case, the output of the classification layer is a probability distribution over the possible conditions, indicating the likelihood of each condition being present in the target node.

In some embodiments, the classification layer is integrated into the overall architecture of the attention-based GNN. The classification layer is appended to the GNN, and the entire model is trained end-to-end using the multi-modal graph database and the corresponding condition labels. The integration allows the GNN and the classification layer to learn and optimize their parameters jointly, enabling the model to capture the relevant features and relationships in the graph that are predictive of the conditions.

In some embodiments, the classification layer is designed to distinguish between rare and common diseases based on the learned features and relationships within the multi-modal graph database. Rare diseases often have unique characteristics and associations that may be difficult to capture using traditional classification approaches. By leveraging the rich information present in the multi-modal graph database and the attention mechanism of the GNN, the classification layer identifies patterns and discriminative features that are indicative of rare diseases. The model learns, or is trained, to assign higher importance to the relevant nodes and edges in the graph that are associated with rare diseases, enabling more accurate prediction and identification of these conditions.

By applying the embedding data object to a classification layer, the method 200 enables the prediction of one or more conditions for the target node based on the learned representations and relationships in the multi-modal graph database. The classification layer leverages the informative embeddings generated by the attention-based GNN to make accurate and reliable predictions, taking into account the complex interactions and dependencies within the graph. The integration of the classification layer with the GNN allows for end-to-end training and optimization, enhancing the overall performance and generalizability of the condition identification model.

In some embodiments, at step 290, the method 200 includes generating, by the one or more processors and based on the application of the embedding object to the classification layer, the probability of one or more predicted conditions appearing in the target node. The output of the classification layer is a probability distribution over the possible conditions, indicating the likelihood of each condition being present in the target node. In some embodiments, the probabilities are generated by one or more function, such as a softmax activation function applied to one or more output of the classification layer, which normalizes the output values to sum up to one. The predicted conditions and their associated probabilities provide valuable insights into the potential health risks and concerns for the patient or member represented by the target node. Healthcare providers can use this information to make informed decisions regarding further diagnostic tests, treatment plans, or preventive measures. The probabilities can also be used to prioritize and allocate resources effectively, focusing on the conditions that are most likely to impact the patient's health outcomes.

In some embodiments, the method 200 further includes initiating, by the one or more processors, the performance of one or more actions based on the generated probabilities of the predicted conditions. The one or more actions may include, but are not limited to, transmitting a message to a healthcare provider or patient, displaying a message on a user interface, automatically generating one or more recommendations, initiating one or more interventions, forming one or more treatment plans, and providing an indication of one or more genetic tests. For example, if the method 200 predicts a high probability of a rare condition for a patient, it may automatically send an alert to the patient's healthcare provider, prompting them to review the patient's case and consider appropriate diagnostic tests or treatment options. Additionally, the method 200 may generate personalized treatment recommendations based on the predicted conditions and the patient's specific characteristics, such as age, gender, and medical history. These recommendations can be displayed on a user interface accessible to healthcare providers, enabling them to make informed decisions and initiate timely interventions. Furthermore, the method 200 may identify potential genetic factors contributing to the predicted conditions and suggest relevant genetic tests to confirm the diagnosis or guide treatment selection. By automating these actions based on the output of the condition identification model, the method 200 can facilitate early detection, accurate diagnosis, and personalized management of various health conditions, improving patient outcomes and healthcare efficiency.

FIG. 3 is a diagram of an exemplary member graph network 300, according to some embodiments of the present disclosure. As shown in FIG. 3, the member graph network 300 includes various types of nodes, each representing a specific aspect of a patient's healthcare data. The member graph network 300 may include, but is not limited to, nodes such as:
Patient node 305: Contains patient information, such as name, age, gender, and one or more unique identifiers for the patient.
Symptom node 310: Represents a specific symptom experienced by the patient, such as chest pain, fever, or headache, and/or represents a plurality of symptoms.
Conditions node 315: Represents one or more diagnosed condition or disease, such as diabetes, hypertension, or asthma.
Medication node 320: Represents one or more medications prescribed to the patient, including dosage and frequency information.
Encounter node 325: Represents details of one or more healthcare visits or interactions, such as date, location, and type of service provided.
Lab diagnostics node 330: Represents one or more details about diagnostic tests and their results, such as blood tests, imaging studies, or biopsies.
Providers node 335: Represents data associated with one or more healthcare professionals involved in the patient's care, including physicians, nurses, and specialists.
Attribute node 340: Represents one or more demographic and lifestyle factors, such as smoking status, family history, and socioeconomic indicators.
Code node 345: Represents one or more standardized medical codes, such as SNOMED or ICD-10, which classify and describe medical conditions, procedures, and treatments.

In the member graph network 300, each node may include one or more connections to other nodes, representing the relationships and associations between different aspects of the patient's healthcare data. For example, a symptom node 310 may be connected to patient node 305, indicating the patient's self-reporting of symptoms and severity. Similarly, a medication node 320 may be linked to both a patient node 305 and a conditions node 315 (by way of code node 345), signifying that the medication is prescribed for a specific patient to treat a particular condition. It is important to note that the configuration shown in FIG. 3 is just one possible representation of a member graph network, and other configurations may exist within the scope of the present disclosure. Furthermore, each node type (e.g., symptom node 310) may represent a single node containing multiple indicators or be broken down into multiple nodes, each representing a specific indicator. For instance, there could be multiple symptom nodes 310, each corresponding to a different symptom experienced by the patient. This flexibility in node representation allows for a more granular and comprehensive modeling of the patient's healthcare data within the member graph network 300.

The member graph network 300 is a dynamic, evolving structure that is periodically updated to incorporate new data as a patient's healthcare journey progresses. One trigger for updating the member graph network 300 is when a member has a new encounter, such as a visit to a healthcare provider, a hospital stay, or a telemedicine consultation. During these encounters, new information is generated and collected, including symptoms reported, diagnoses made, medications prescribed, tests performed, and treatments administered. This new data is then integrated into the member graph network 300, creating new nodes and edges or updating the attributes of existing nodes and the weights of the connections between them. Through this continuous updating process, the member graph network 300 hosts not only the individual's intrinsic characteristics but also their cumulative healthcare experiences and exposures spanning multiple encounters, lab tests, imaging studies, treatments, vital signs, and interactions with various healthcare providers. To ensure a comprehensive and accurate representation of the patient's health status, data is sourced from diverse databases, including (1) EHR databases containing detailed clinical information; (2) administrative databases encompassing medical and pharmacy claims, lab results, and membership data; (3) rare disease global databases providing information on uncommon conditions; (4) disease-symptom network databases capturing the relationships between different conditions and their associated symptoms; and (5) drug-disease network databases highlighting the interactions between medications and the conditions they are used to treat.

FIG. 4 is a diagram of an exemplary disease graph network 400, according to some embodiments of the present disclosure. As shown in FIG. 4, the disease graph network 400 is a complex, interconnected structure that represents the relationships between various biological and medical entities:
Symptom node 410: Represents one or more symptoms or clinical manifestations, such as fever, pain, or fatigue.
Conditions node 415: Represents one or more diseases or medical conditions, such as diabetes, cancer, or Alzheimer's disease.
Medication node 420: Represents one or more drugs or therapeutic agents used to treat or manage a condition, such as insulin, chemotherapy, or antidepressants.
Protein node 425: Represents one or more proteins or biomolecules involved in the pathophysiology or treatment of a condition, such as a receptor, enzyme, or antibody.

In the disease graph network 400, the nodes are connected by edges that represent the relationships and interactions between them. Protein nodes 425 are connected to conditions nodes 415 and medication nodes 420, indicating their involvement in the disease process or treatment mechanism. For example, a protein node 425 may represent a receptor that is targeted by a specific medication node 420 to treat a particular condition node 415. Symptom nodes 410 are connected to conditions nodes 415 and medication nodes 420, signifying the clinical manifestations associated with a disease or the side effects of a treatment. Conditions nodes 415 and medication nodes 420 are connected to all other node types, reflecting the complex interplay between diseases, treatments, symptoms, and biological pathways.

In addition to these connections, protein nodes 425 are also linked to one or more additional protein nodes 425, representing protein-protein interactions. These interactions may include physical binding, functional cooperation, or regulatory relationships between proteins. By capturing these protein-protein interactions, the disease graph network 400 provides a more comprehensive view of the underlying biological mechanisms driving disease processes and treatment responses.

It is important to note that the configuration shown in FIG. 4 is just one possible representation of a disease graph network, and other configurations may exist within the scope of the present disclosure. The disease graph network 400 is a dynamic, evolving structure that is periodically updated to incorporate new scientific knowledge and medical insights. Data is sourced from various databases, including scientific literature, clinical trial results, omics databases, and curated knowledge bases, among others.

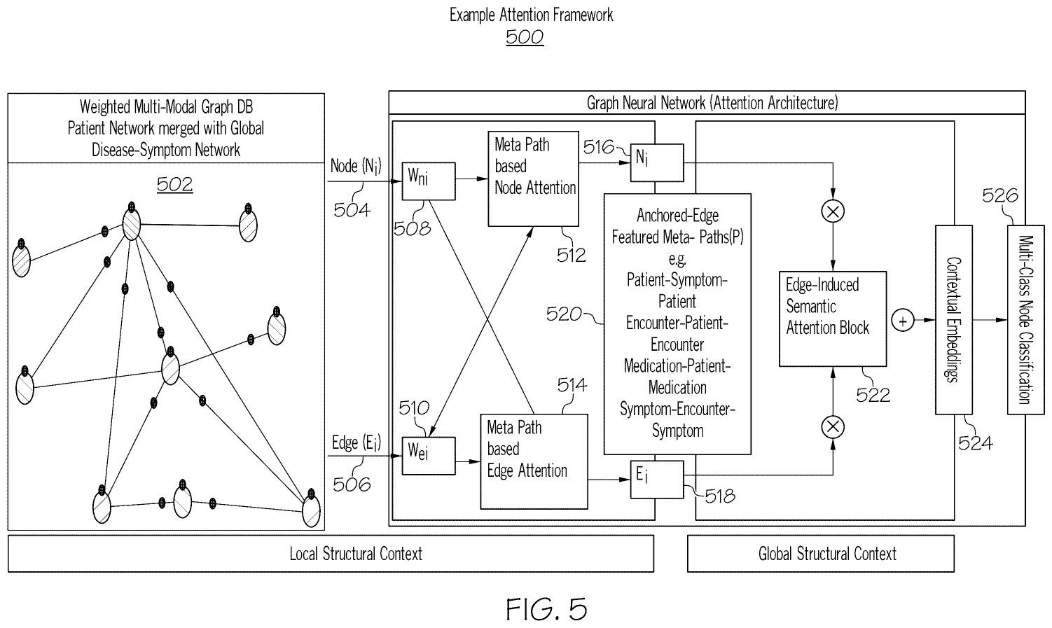

FIG. 5 is a diagram of example components of an attention framework, according to some embodiments of the disclosure. FIG. 5 illustrates an attention framework 500 for processing and analyzing the weighted multi-modal graph database 502, which represents the merged patient graph network and the disease graph network. The attention framework 500 leverages graph neural network (GNN) techniques to learn and update node and edge representations, enabling more accurate and context-aware predictions of patient conditions.

In some embodiments, the weighted multi-modal graph database 502, generated by the merging of the patient graph network and the drug graph network, serves as the input to the attention framework 500. It consists of nodes 504 (represented as Nᵢ) and edges 506 (represented as Eⱼ). Each node 504 represents an entity in the graph, such as a patient, symptom, condition, medication, or protein, along with its associated attributes. Each edge 506 represents a relationship or interaction between two nodes, such as a patient experiencing a symptom, a medication treating a condition, or a protein interacting with another protein. The nodes 504 and edges 506 have initial weights, denoted as node weights 508 (W_{Ni}) and edge weights 510 (W_{Ej}), respectively. These weights reflect the importance or strength of the nodes and edges in the context of the graph.

The attention framework 500 includes a meta-path-based node attention block 512 and a meta-path-based edge attention block 514. These blocks leverage the concept of meta-paths, which are predefined sequences of node and edge types that capture specific semantic relationships within the graph. Meta-paths provide a way to incorporate domain knowledge and guide the attention mechanism to focus on relevant node and edge features.

The meta-path-based node attention block 512 takes the initial node features and edge information as input and applies an attention mechanism to compute updated node representations, denoted as Nᵢ′ 516. The attention mechanism is trained to assign different weights to the neighboring nodes and edges based on their relevance to the target node, as determined by the meta-paths. By aggregating the weighted contributions of the neighboring nodes and edges, the node attention block 512 generates more informative and context-aware node representations.

Similarly, the meta-path-based edge attention block 514 takes the initial node and edge features as input and applies an attention mechanism to compute updated edge representations, denoted as Eⱼ′ 518. The edge attention mechanism is trained to assign different weights to the connected nodes and their attributes based on the relevance of the edge in the context of the meta-paths. By incorporating the weighted contributions of the connected nodes and their attributes, the edge attention block 514 generates more informative and context-aware edge representations.

In addition to the learned node and edge representations, the attention framework 500 also considers anchored-edge feature meta-paths 520. These are predefined meta-paths that capture important and stable relationships within the graph, such as patient-symptom-patient, encounter-patient-encounter, medication-patient-medication, and symptom-encounter-symptom. In some embodiments, the weights and attributes associated with these anchored meta-paths are not significantly modified during the learning process, as they represent fundamental domain knowledge that should be preserved and guide the development and adjustment of weights and attributes associated with one or more other path.

The updated node representations (Nᵢ′ 516), updated edge representations (Eⱼ′ 518), and anchored-edge feature meta-paths 520 are then combined in the edge-induced semantic attention block 522. This block applies another level of attention to learn the importance of different edge types and their associated meta-paths in the context of the specific node and its neighbors. By attending to the most informative edge types and meta-paths, the semantic attention block 522 generates highly expressive and context-aware node representations, referred to as contextual embeddings 524.

The contextual embeddings 524 capture the rich semantic information present in the weighted multi-modal graph database 502, considering the complex interactions between nodes, edges, and their associated meta-paths. These embeddings serve as input to the multi-class node classification module 526, which predicts the probabilities of one or more conditions being present for the condition node of interest.

The contextual embeddings 524 generated by the attention framework 500 can take various technical forms and data structures, depending on the specific implementation and requirements of the system. In general, the contextual embeddings are represented as high-dimensional vectors or tensors that encode the semantic information and relationships captured from the weighted multi-modal graph database 502. These embeddings can be stored and manipulated using standard data structures such as arrays, matrices, or sparse matrices, depending on the size and sparsity of the graph. In some embodiments, the contextual embeddings are represented as dense vectors, where each dimension corresponds to a learned feature or attribute. In other embodiments, the embeddings are represented as sparse vectors or matrices, where only the non-zero elements are stored to reduce memory consumption and computational complexity. The choice of data structure for the contextual embeddings depends on factors such as the dimensionality of the embeddings, the number of nodes in the graph, and the available computational resources. Furthermore, the contextual embeddings may be stored in various formats, such as CSV (Comma-Separated Values), JSON (JavaScript Object Notation), or HDF5 (Hierarchical Data Format), to facilitate data exchange and interoperability with other components of the system, such as the multi-class node classification module 526.

In some embodiments, the multi-class node classification module 526 is implemented as a feedforward neural network or a multi-layer perceptron (MLP). It intakes the contextual embeddings 524 as input and applies a series of linear transformations and non-linear activations to compute the output probabilities. The classification module 526 is trained using labeled data, where each training sample consists of a contextual embedding and its corresponding ground-truth condition labels. The training process involves optimizing the parameters of the classification module to minimize a loss function, such as cross-entropy loss, which measures the discrepancy between the predicted probabilities and the true condition labels.

In some embodiments, the training of the multi-class node classification module 526 can be performed independently or jointly with the entire attention framework 500. In the independent training approach, the contextual embeddings 524 are first generated using the attention framework 500, and then used as input to train the classification module 526 separately. This approach allows for more flexibility and modularity, as different classification models can be experimented with or optimized without modifying the attention framework. In the joint training approach, the parameters of the attention framework 500 and the classification module 526 are learned simultaneously, allowing for end-to-end optimization. This approach enables the attention framework to learn embeddings that are directly optimized for the classification task, potentially leading to improved performance. The choice between independent and joint training depends on factors such as the complexity of the graph, the availability of labeled data, and the computational resources.

In some embodiments, the training data for the multi-class node classification module 526 typically consists of labeled examples, where each example includes a contextual embedding 524 and its corresponding ground-truth condition labels. These labels indicate the presence or absence of specific conditions for the corresponding node in the graph. The labeled data can be obtained from various sources, such as electronic health records (EHRs), medical claims data, or expert annotations. In some embodiments, the labeled data may be augmented with synthetic examples generated using techniques such as data augmentation or adversarial learning to improve the robustness and generalization of the classification model. Additionally, the training data may be preprocessed to handle imbalances, missing values, or the like.

During inference, given a patient's weighted multi-modal graph, the attention framework 500 processes the graph through the node attention block 512, edge attention block 514, and semantic attention block 522 to generate the contextual embeddings 524. These embeddings are then passed through the trained multi-class node classification module 526 to predict the probabilities of various conditions being present for the patient. In some embodiments, the attention framework is trained 500 is trained specifically for predicting rare conditions, rather than all possible conditions. Rare conditions are typically defined as diseases or disorders that affect a small percentage of a target population, for example conditions that impact less than 200,000 patients in the United States. Training the attention framework 500 to focus on rare conditions can be particularly beneficial, as these conditions are often difficult to diagnose due to their low prevalence and the limited availability of expert knowledge and labeled data. By tailoring the attention framework to prioritize the identification of rare conditions, the system can help healthcare providers identify patients with these conditions more accurately and efficiently. In this scenario, the multi-class node classification module 526 is trained using labeled data that primarily consists of examples with rare conditions, enabling it to specialize in distinguishing between different rare conditions based on the contextual embeddings 524 generated by the attention framework. This focused approach can lead to improved detection and management of rare conditions, benefiting patients who may otherwise face delayed or misdiagnosed conditions.

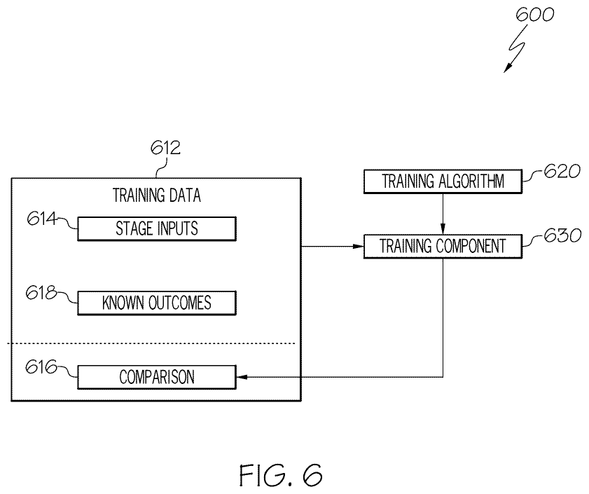

One or more implementations disclosed herein include and/or are implemented using a machine-learning model. For example, one or more of the modules of the condition identification platform 120 are implemented using a machine-learning model and/or are used to train the machine-learning model. FIG. 6 shows an example machine-learning training flow chart, according to some embodiments of the disclosure. Referring to FIG. 6, a given machine-learning model is trained using the training flow chart 600. The training data 612 includes one or more of stage inputs 614 and the known outcomes 618 related to the machine-learning model to be trained. The stage inputs 614 are from any applicable source including text, visual representations, data, values, comparisons, and stage outputs, e.g., one or more outputs from one or more steps from FIG. 2. The known outcomes 618 are included for the machine-learning models generated based on supervised or semi-supervised training, or can based on known labels, such as topic labels. An unsupervised machine-learning model is not trained using the known outcomes 618. The known outcomes 618 includes known or desired outputs for future inputs similar to or in the same category as the stage inputs 614 that do not have corresponding known outputs.

The training data 612 and a training algorithm 620, e.g., one or more of the modules implemented using the machine-learning model and/or are used to train the machine-learning model, is provided to a training component 630 that applies the training data 612 to the training algorithm 620 to generate the machine-learning model. According to an implementation, the training component 630 is provided comparison results 616 that compare a previous output of the corresponding machine-learning model to apply the previous result to re-train the machine-learning model. The comparison results 616 are used by the training component 630 to update the corresponding machine-learning model. The training algorithm 620 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIG. 2 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

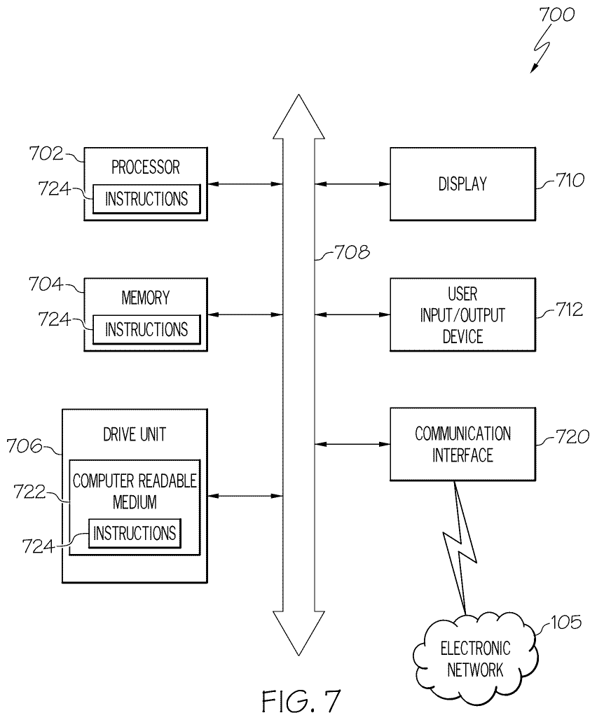

FIG. 7 illustrates an implementation of a computer system that executes techniques presented herein. The computer system 700 includes a set of instructions that are executed to cause the computer system 700 to perform any one or more of the methods or computer based functions disclosed herein. The computer system 700 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 700 operates in the capacity of a server or as a client user computer in a server-client user environment, or as a peer computer system in a peer-to-peer (or distributed) environment. The computer system 700 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 700 is implemented using electronic devices that provide voice, video, or data communication. Further, while the computer system 700 is illustrated as a single system, the term “system” shall also be taken to include any collection of systems or sub-systems that individually or jointly execute a set, or multiple sets, of instructions to perform one or more computer functions.

As illustrated in FIG. 7, the computer system 700 includes a processor 702, e.g., a central processing unit (CPU), a graphics processing unit (GPU), or both. The processor 702 is a component in a variety of systems. For example, the processor 702 is part of a standard personal computer or a workstation. The processor 702 is one or more processors, digital signal processors, application specific integrated circuits, field programmable gate arrays, servers, networks, digital circuits, analog circuits, combinations thereof, or other now known or later developed devices for analyzing and processing data. The processor 702 implements a software program, such as code generated manually (i.e., programmed).

The computer system 700 includes a memory 704 that communicates via bus 708. The memory 704 is a main memory, a static memory, or a dynamic memory. The memory 704 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 704 includes a cache or random-access memory for the processor 702. In alternative implementations, the memory 704 is separate from the processor 702, such as a cache memory of a processor, the system memory, or other memory. The memory 704 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 704 is operable to store instructions executable by the processor 702. The functions, acts, or tasks illustrated in the figures or described herein are performed by the processor 702 executing the instructions stored in the memory 704. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 700 further includes a display 710, such as a liquid crystal display (LCD), an organic light emitting diode (OLED), a flat panel display, a solid-state display, a cathode ray tube (CRT), a projector, a printer or other now known or later developed display device for outputting determined information. The display 710 acts as an interface for the user to see the functioning of the processor 702, or specifically as an interface with the software stored in the memory 704 or in the drive unit 706.

Additionally or alternatively, the computer system 700 includes an input/output device 712 configured to allow a user to interact with any of the components of the computer system 700. The input/output device 712 is a number pad, a keyboard, a cursor control device, such as a mouse, a joystick, touch screen display, remote control, or any other device operative to interact with the computer system 700.

The computer system 700 also includes the drive unit 706 implemented as a disk or optical drive. The drive unit 706 includes a computer-readable medium 722 in which one or more sets of instructions 724, e.g. software, is embedded. Further, the sets of instructions 724 embodies one or more of the methods or logic as described herein. The sets of instructions 724 resides completely or partially within the memory 704 and/or within the processor 702 during execution by the computer system 700. The memory 704 and the processor 702 also include computer-readable media as discussed above.

In some systems, computer-readable medium 722 includes the set of instructions 724 or receives and executes the set of instructions 724 responsive to a propagated signal so that a device connected to network 105 communicates voice, video, audio, images, or any other data over the network 105. Further, the sets of instructions 724 are transmitted or received over the network 105 via the communication port or interface 720, and/or using the bus 708. The communication port or interface 720 is a part of the processor 702 or is a separate component. The communication port or interface 720 is created in software or is a physical connection in hardware. The communication port or interface 720 is configured to connect with the network 105, external media, the display 710, or any other components in the computer system 700, or combinations thereof. The connection with the network 105 is a physical connection, such as a wired Ethernet connection, or is established wirelessly as discussed below. Likewise, the additional connections with other components of the computer system 700 are physical connections or are established wirelessly. The network 105 alternatively be directly connected to the bus 708.

While the computer-readable medium 722 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 722 is non-transitory, and may be tangible.

The computer-readable medium 722 includes a solid-state memory such as a memory card or other package that houses one or more non-volatile read-only memories. The computer-readable medium 722 is a random-access memory or other volatile re-writable memory. Additionally or alternatively, the computer-readable medium 722 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 700 is connected to the network 105. The network 105 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 105 includes wide area networks (WAN), such as the Internet, local area networks (LAN), campus area networks, metropolitan area networks, a direct connection such as through a Universal Serial Bus (USB) port, or any other networks that allows for data communication. The network 105 is configured to couple one computing device to another computing device to enable communication of data between the devices. The network 105 is generally enabled to employ any form of machine-readable media for communicating information from one device to another. The network 105 includes communication methods by which information travels between computing devices. The network 105 is divided into sub-networks. The sub-networks allow access to all of the other components connected thereto or the sub-networks restrict access between the components. The network 105 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the instructions for carrying out such a method or element of a method forms a means for carrying out the method or element of a method. Furthermore, an element described herein of an apparatus embodiment is an example of a means for carrying out the function performed by the element for the purpose of carrying out the disclosure.

In the description provided herein, numerous specific details are set forth. However, it is understood that embodiments of the disclosure are practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognize that other and further modifications are made thereto without departing from the spirit of the disclosure, and it is intended to claim all such changes and modifications as falling within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

Example 1. A computer-implemented method comprising; receiving, by one or more processors, a member data object associated with a member, the member data object including one or more indicators, each indicator having an associated dimension; accessing, by the one or more processors, a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modifying, by the one or more processors, one or more of the plurality of nodes and one or more edges therebetween of the member-specific graph network based on the one or more indicators and the one or more respective dimensions of the member data object; generating, by the one or more processors, a multi-modal graph database by combining the modified member-specific graph network and a disease graph network; applying, by the one or more processors, the multi-modal graph database to an attention-based graph neural network (GNN) trained to identify associations between nodes by learning to dynamically allocate attention weights to edges of the multi-modal graph database; generating, by the one or more processors and based on the applying of the multi-modal graph database to the attention-based GNN, an embedding data object, the embedding data object including a plurality of node identifiers each uniquely representing a node in the multi-modal graph database and a plurality of vectors each corresponding to a node identifier and representing one or more features of the node and one or more relationships of the node within the multi-modal graph database; selecting, by the one or more processors, a target node from of the plurality of nodes, the target node associated with condition data; applying, by the one or more processors, the embedding data object to a classification layer, the classification layer trained to output one or more predicted conditions for the target node based on one or more input embeddings; and generating, by the one or more processors and based on the applying of the embedding object to the classification layer, a probability of one or more predicted conditions appearing in the target node.

Example 2. The computer-implemented method of example 1, wherein the one or more indicators comprise one or more health-related indicators collected during a member data collection event.

Example 3. The computer-implemented method of example 2, wherein the one or more health-related indicators comprise text data and image data.

Example 4. The computer-implemented method of any of examples 1-3, further comprising: initiating, by the one or more processors, a performance of one or more actions.

Example 5. The computer-implemented method of example 4, wherein the one or more actions includes one or more of: transmitting a message to a healthcare provider or patient, displaying a message on a user interface, automatically generating one or more recommendations, initiating one or more interventions, forming one or more treatment plans; or providing an indication of one or more genetic test.

Example 6. The computer-implemented method of any of examples 1-5, wherein generating a multi-modal graph database by combining the modified member-specific graph network and the disease graph network includes merging the modified member-specific graph network and the disease graph network, the merging comprising: identifying common nodes present in both the modified member-specific graph network and the disease graph network; combining edge information from the modified member-specific graph network and the disease graph network for each common node; aggregating node features of each common node from the modified member-specific graph network and the disease graph network using an aggregation function; and preserving non-common nodes and their associated edge information from the modified member-specific graph network or the disease graph network in the merged graph database.

Example 7. The computer-implemented method of any of examples 1-6, further comprising: utilizing, by the one or more processors, a GNN-based entity resolution technique to resolve node ambiguity during the combining of the modified member-specific graph network and the disease graph network, wherein the entity resolution technique employs similarity metrics to identify and merge duplicate nodes across the modified member-specific graph network and the disease graph network.

Example 8. The computer-implemented method of any of examples 1-7, wherein the classification layer is provided within the attention-based GNN, wherein the classification layer is configured to distinguish between rare and common diseases based on learned features and relationships within the multi-modal graph database.

Example 9. The computer-implemented method of any of examples 1-8, further comprising preprocessing, by the one or more processors, the member data object and the disease graph network data prior to modifying the member-specific graph network, wherein preprocessing includes one or more of: data normalization, entity resolution, or missing data imputation.

Example 10. The computer-implemented method of any of examples 1-9, wherein the attention-based GNN is configured to perform weight prediction on missing edge weights within the multi-modal graph database, wherein the prediction includes employing a loss function based at least in part on one or more default and absent edge weights.

Example 11. A system comprising memory and one or more processors communicatively coupled to the memory, the one or more processors configured to: receive a member data object associated with a member, the member data object including one or more indicators, each indicator having an associated dimension; access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modify one or more of the plurality of nodes and one or more edges therebetween of the member-specific graph network based on the one or more indicators and the one or more respective dimensions of the member data object; generate a multi-modal graph database by combining the modified member-specific graph network and a disease graph network; apply the multi-modal graph database to an attention-based graph neural network (GNN) trained to identify associations between nodes by learning to dynamically allocate attention weights to edges of the multi-modal graph database; generate, based on the applying of the multi-modal graph database to the attention-based GNN, an embedding data object, the embedding data object including a plurality of node identifiers each uniquely representing a node in the multi-modal graph database and a plurality of vectors each corresponding to a node identifier and representing one or more features of the node and one or more relationships of the node within the multi-modal graph database; select a target node from of the plurality of nodes, the target node associated with condition data; apply the embedding data object to a classification layer, the classification layer trained to output one or more predicted conditions for the target node based on one or more input embeddings; and generate, based on the applying of the embedding object to the classification layer, a probability of one or more predicted conditions appearing in the target node.

Example 12. The system of example 11, wherein the one or more indicators comprise one or more health-related indicators collected during a member data collection event.

Example 13. The system of example 12, wherein the one or more health-related indicators comprise text data and image data.

Example 14. The system of any of examples 11-13, wherein the one or more processors are further configured to: initiate a performance of one or more actions.

Example 15. The system of example 14, wherein the one or more actions includes one or more of: transmitting a message to a healthcare provider or patient, displaying a message on a user interface, automatically generating one or more recommendations, initiating one or more interventions, forming one or more treatment plans; or providing an indication of one or more genetic test.

Example 16. The system of any of examples 11-15, wherein generating the multi-modal graph database by combining the modified member-specific graph network and the disease graph network includes merging the modified member-specific graph network and the disease graph network, the merging comprising: identifying common nodes present in both the modified member-specific graph network and the disease graph network; combining edge information from the modified member-specific graph network and the disease graph network for each common node; aggregating node features of each common node from the modified member-specific graph network and the disease graph network using an aggregation function; and preserving non-common nodes and their associated edge information from the modified member-specific graph network or the disease graph network in the merged graph database.

Example 17. The system of any of examples 11-16, wherein the one or more processors are further configured to: utilize a GNN-based entity resolution technique to resolve node ambiguity during the combining of the modified member-specific graph network and the disease graph network, wherein the entity resolution technique employs similarity metrics to identify and merge duplicate nodes across the modified member-specific graph network and the disease graph network.

Example 18. The system of any of examples 11-17, wherein the classification layer is provided within the attention-based GNN, wherein the classification layer is configured to distinguish between rare and common diseases based on learned features and relationships within the multi-modal graph database.

Example 19. The system of any of examples 11-18, wherein the one or more processors are further configured to: preprocess the member data object and the disease graph network data prior to modifying the member-specific graph network, wherein preprocessing includes one or more of: data normalization, entity resolution, or missing data imputation.

Example 20. One or more non-transitory computer-readable storage media including instructions that, when executed by one or more processors, cause the one or more processors to: receive a member data object associated with a member, the member data object including one or more indicators, each indicator having an associated dimension; access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto; modify one or more of the plurality of nodes and one or more edges therebetween of the member-specific graph network based on the one or more indicators and the one or more respective dimensions of the member data object; generate a multi-modal graph database by combining the modified member-specific graph network and a disease graph network; apply the multi-modal graph database to an attention-based graph neural network (GNN) trained to identify associations between nodes by learning to dynamically allocate attention weights to edges of the multi-modal graph database; generate, based on the applying of the multi-modal graph database to the attention-based GNN, an embedding data object, the embedding data object including a plurality of node identifiers each uniquely representing a node in the multi-modal graph database and a plurality of vectors each corresponding to a node identifier and representing one or more features of the node and one or more relationships of the node within the multi-modal graph database; select a target node from of the plurality of nodes, the target node associated with condition data; apply the embedding data object to a classification layer, the classification layer trained to output one or more predicted conditions for the target node based on one or more input embeddings; and generate, based on the applying of the embedding object to the classification layer, a probability of one or more predicted conditions appearing in the target node.

What is claimed is:

1. A computer-implemented method comprising;
receiving, by one or more processors, a member data object associated with a member, the member data object including one or more indicators, each indicator having an associated dimension;
accessing, by the one or more processors, a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto;
modifying, by the one or more processors, one or more of the plurality of nodes and one or more edges therebetween of the member-specific graph network based on the one or more indicators and the one or more respective dimensions of the member data object;
generating, by the one or more processors, a multi-modal graph database by combining the modified member-specific graph network and a disease graph network;
applying, by the one or more processors, the multi-modal graph database to an attention-based graph neural network (GNN) trained to identify associations between nodes by learning to allocate attention weights to edges of the multi-modal graph database;
generating, by the one or more processors and based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database;
selecting, by the one or more processors, a target node from the plurality of nodes, the target node associated with condition data;
applying, by the one or more processors, the embedding data object to a classification layer, the classification layer trained to output one or more predicted conditions for the target node based on one or more input embeddings; and
generating, by the one or more processors and based on the applying of the embedding object to the classification layer, a probability of one or more predicted conditions appearing in the target node.

2. The computer-implemented method of claim 1, wherein the one or more indicators comprise one or more health-related indicators collected during a member data collection event.

3. The computer-implemented method of claim 2, wherein the one or more health-related indicators comprise text data and image data.

4. The computer-implemented method of claim 1, further comprising: initiating, by the one or more processors, a performance of one or more actions.

5. The computer-implemented method of claim 4, wherein the one or more actions includes one or more of: transmitting a message to a healthcare provider or patient, displaying a message on a user interface, automatically generating one or more recommendations, initiating one or more interventions, forming one or more treatment plans; or providing an indication of one or more genetic tests.

6. The computer-implemented method of claim 1, wherein generating the multi-modal graph database by combining the modified member-specific graph network and the disease graph network includes merging the modified member-specific graph network and the disease graph network, the merging comprising:
identifying common nodes present in both the modified member-specific graph network and the disease graph network;
combining edge information from the modified member-specific graph network and the disease graph network for each common node;
aggregating node features of each common node from the modified member-specific graph network and the disease graph network using an aggregation function; and
preserving non-common nodes and their associated edge information from the modified member-specific graph network or the disease graph network in the merged graph database.

7. The computer-implemented method of claim 1, further comprising: utilizing, by the one or more processors, a GNN-based entity resolution technique to resolve node ambiguity during the combining of the modified member-specific graph network and the disease graph network, wherein the entity resolution technique employs similarity metrics to identify and merge duplicate nodes across the modified member-specific graph network and the disease graph network.

8. The computer-implemented method of claim 1, wherein the classification layer is provided within the attention-based GNN, wherein the classification layer is configured to distinguish between rare and common diseases based on learned features and relationships within the multi-modal graph database.

9. The computer-implemented method of claim 1, further comprising preprocessing, by the one or more processors, the member data object and the disease graph network data prior to modifying the member-specific graph network, wherein preprocessing includes one or more of: data normalization, entity resolution, or missing data imputation.

10. The computer-implemented method of claim 1, wherein the attention-based GNN is configured to perform weight prediction on missing edge weights within the multi-modal graph database, wherein the prediction includes employing a loss function based at least in part on one or more default and absent edge weights.

11. A system comprising memory and one or more processors communicatively coupled to the memory, the one or more processors configured to:
receive a member data object associated with a member, the member data object including one or more indicators, each indicator having an associated dimension;
access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto;
modify one or more of the plurality of nodes and one or more edges therebetween of the member-specific graph network based on the one or more indicators and the one or more respective dimensions of the member data object;
generate a multi-modal graph database by combining the modified member-specific graph network and a disease graph network;
apply the multi-modal graph database to an attention-based graph neural network (GNN) trained to identify associations between nodes by learning to allocate attention weights to edges of the multi-modal graph database;
generate, based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database;
select a target node from the plurality of nodes, the target node associated with condition data;
apply the embedding data object to a classification layer, the classification layer trained to output one or more predicted conditions for the target node based on one or more input embeddings; and
generate, based on the applying of the embedding object to the classification layer, a probability of one or more predicted conditions appearing in the target node.

12. The system of claim 11, wherein the one or more indicators comprise one or more health-related indicators collected during a member data collection event.

13. The system of claim 12, wherein the one or more health-related indicators comprise text data and image data.

14. The system of claim 11, wherein the one or more processors are further configured to: initiate a performance of one or more actions.

15. The system of claim 14, wherein the one or more actions includes one or more of: transmitting a message to a healthcare provider or patient, displaying a message on a user interface, automatically generating one or more recommendations, initiating one or more interventions, forming one or more treatment plans; or providing an indication of one or more genetic tests.

16. The system of claim 11, wherein generating the multi-modal graph database by combining the modified member-specific graph network and the disease graph network includes merging the modified member-specific graph network and the disease graph network, the merging comprising:
identifying common nodes present in both the modified member-specific graph network and the disease graph network;
combining edge information from the modified member-specific graph network and the disease graph network for each common node;
aggregating node features of each common node from the modified member-specific graph network and the disease graph network using an aggregation function; and
preserving non-common nodes and their associated edge information from the modified member-specific graph network or the disease graph network in the merged graph database.

17. The system of claim 11, wherein the one or more processors are further configured to: utilize a GNN-based entity resolution technique to resolve node ambiguity during the combining of the modified member-specific graph network and the disease graph network, wherein the entity resolution technique employs similarity metrics to identify and merge duplicate nodes across the modified member-specific graph network and the disease graph network.

18. The system of claim 11, wherein the classification layer is provided within the attention-based GNN, wherein the classification layer is configured to distinguish between rare and common diseases based on learned features and relationships within the multi-modal graph database.

19. The system of claim 11, wherein the one or more processors are further configured to: preprocess the member data object and the disease graph network data prior to modifying the member-specific graph network, wherein preprocessing includes one or more of: data normalization, entity resolution, or missing data imputation.

20. One or more non-transitory computer-readable storage media including instructions that, when executed by one or more processors, cause the one or more processors to:
receive a member data object associated with a member, the member data object including one or more indicators, each indicator having an associated dimension;
access a member-specific graph network, the member-specific graph network having a plurality of nodes representing respective member attributes, each node connected to one or more other nodes by one or more respective edges, each edge indicative of an association between the nodes connected thereto, and each edge including an edge weight indicative of a relative importance of the association between the nodes connected thereto;
modify one or more of the plurality of nodes and one or more edges therebetween of the member-specific graph network based on the one or more indicators and the one or more respective dimensions of the member data object;
generate a multi-modal graph database by combining the modified member-specific graph network and a disease graph network;
apply the multi-modal graph database to an attention-based graph neural network (GNN) trained to identify associations between nodes by learning to allocate attention weights to edges of the multi-modal graph database;
generate, based on the applying of the multi-modal graph database to the attention-based GNN, an embedding data object, the embedding data object including a plurality of node identifiers each representing a node in the multi-modal graph database and a plurality of vectors each corresponding to a node identifier and representing one or more features of the node and one or more relationships of the node within the multi-modal graph database;
select a target node from the plurality of nodes, the target node associated with condition data;
apply the embedding data object to a classification layer, the classification layer trained to output one or more predicted conditions for the target node based on one or more input embeddings; and
generate, based on the applying of the embedding object to the classification layer, a probability of one or more predicted conditions appearing in the target node.
